(19) United States
(12) Patent Application Publication
(10) Pub. No.: US 2025/0228576 A1
(43) Pub. Date: Jul. 17, 2025

(54) INTRAVASCULAR ULTRASOUND TRANSDUCERS ENABLED TISSUE ABLATION FOR TREATMENT OF IN-STENT RESTENOSIS

(71) Applicant: North Carolina State University, Raleigh, NC (US)

(72) Inventors: Xiaoning Jiang, Cary, NC (US); Howuk Kim, San Jose, CA (US); Xuming Dai, Roslyn, NY (US)

(21) Appl. No.: 18/849,465

(22) PCT Filed: Mar. 21, 2023
(86) PCT No.: PCT/US2023/015794

§ 371 Date: Sep. 20, 2024

Related U.S. Application Data
(60) Provisional application No. 63/322,632, filed on Mar. 22, 2022.

(51) Int. Cl. A61B 17/22; A61B 17/00; A61F 2/82

(52) U.S. Cl. CPC A61B 17/2202; A61B 2017/00402; A61B 2017/22008; A61B 2017/22027; A61B 2017/22082; A61F 2/82; A61F 2250/0067; A61F 2250/0095

(57) ABSTRACT

A device for therapeutic ablative treatment of residual plaque on, in, and/or surrounding a stent within a blood vessel of a subject includes a jacket configured for insertion within a stent within a blood vessel of a subject. The device further includes an ultrasound transducer located within the jacket and having at least one active element oriented to deliver ultrasound energy in a radial and/or axial direction of the jacket from within the stent to ablate the residual plaque.

### PRIORITY CLAIM

This application claims the priority benefit of U.S. Provisional Patent Application Ser. No. 63/322,632 filed Mar. 22, 2022, the disclosure of which is incorporated herein by reference in its entirety.

### GOVERNMENT INTEREST

This invention was made with government support under grant number HL141967 awarded by the National Institutes of Health. The government has certain rights in the invention.

### BACKGROUND

In-stent restenosis (ISR) refers to luminal re-narrowing within the stent over time (typically 6 to 9 months later). Table 1 shows factors for restenosis and that residual plaque is one of the most predominant factors causing ISR. However, it has been challenging to effectively remove residual plaque within a stent after the treatment due to technical limitations. For instance, FIG. 1 illustrates the stenting procedure of an occluded coronary vessel, where a significant amount of plaque is compressed and remained within the vessel lumen. To be specific, once the stent is inflated inside the artery, plaque is compressed between the vessel wall and the struts, leaving the residual plaque in the side wall of the vessel, as shown in the left-most image in FIG. 1. ISR can then be caused by aggressive neointimal proliferation over the residual plaques. Hence, either reduction or ablation of in-stent neointimal tissue may substantially suppress potential of the ISR.

A few clinical options have been introduced for ISR treatment, yet the existing modalities have their own technical and biomedical limitations. Plain old balloon angioplasty (POBA) is hard to achieve a wide luminal gain due to the elastic recoil of ISR tissues. Cutting balloon angioplasty (CBA), disrupting ISR tissue using micro-blades in the balloon, does not represent a significant improvement in angiographic restenosis in comparison with POBA. CBA is known to have technical limitations, however, leaving residual ISR tissue even after the treatment. Angiographic outcomes with Drug-coated balloons (DCB), delivering anti-proliferative drugs to a target ISR region, were slightly lower than stenting methods. Repeat stenting with drug-eluting stents (DES) is one of the most commonly used and most effective modalities for the treatment of ISR. However, multiple layers of stent struts limit the available lumen area for blood flow. Therefore, in a small caliber vessel or vessel with pre-existing multiple layers of stent, repeating stenting is not feasible. Applications of debulking techniques, such as excimer laser, directional/rotational atherectomy, widening lumen by removal of atheromatous material, are restricted due to vessel wall invasiveness and contact, high operating costs, and maintenance requirements. Lastly, directional/rotational atherectomy still presents a risk of vessel damage during the ISR treatment.

### SUMMARY

A device for therapeutic ablative treatment of residual plaque on, in, or surrounding a stent within a blood vessel of a subject includes a jacket configured for insertion within a stent within a blood vessel of the subject. The device further includes an ultrasound transducer located within the jacket and having at least one active element oriented to deliver ultrasound energy in a radial direction of the jacket from within the stent to ablate residual plaque located on, in, or surrounding the stent.

According to another aspect of the subject matter described herein, the ultrasound transducer comprises one or more piezoelectric elements.

According to another aspect of the subject matter described herein, the one or more piezoelectric elements comprise a plurality of stacks of piezoelectric material, each stack forming a pillar, and the pillars forming a multi-pillar piezoelectric stack (MPPS) device.

According to another aspect of the subject matter described herein, each of the pillars of the MPPS device is separated from adjacent pillars by a gap for reducing lateral vibrational coupling between adjacent pillars. According to another aspect of the subject matter described herein, each of the gaps is at least partially filled with an epoxy resin.

According to another aspect of the subject matter described herein, each of the piezoelectric elements comprises at least one active layer.

According to another aspect of the subject matter described herein, the MPPS device comprises a common backing layer and a common matching layer, between which the at least one active layer of each of the piezoelectric elements is positioned.

According to another aspect of the subject matter described herein, the device comprises a cooling circuit for cooling the ultrasound transducer, the cooling circuit comprising an inlet tube for providing a flow of a fluid into the jacket and an outlet tube for providing a flow of the fluid out of the jacket after the fluid has been heated by the ultrasound transducer during cooling the ultrasound transducer.

According to another aspect of the subject matter described herein, the device comprises an imaging sensor for imaging an area external to the jacket for positioning the device adjacent to a target region within the blood vessel of the subject.

According to another aspect of the subject matter described herein, the sensor comprises an ultrasound imaging sensor.

According to another aspect of the subject matter described herein,
the stent is a metallic stent and the residual plaque is at least partially within a lumen of the metallic stent and the jacket comprises a catheter.

According to another aspect of the subject matter described herein, the ultrasound transducer is configured to receive a signal via a wired or wireless connection for controlling one or more aspects of the ultrasound energy emitted from the ultrasound transducer.

According to another aspect of the subject matter described herein,
the jacket comprises a primary lumen and a secondary lumen that each extend longitudinally within the jacket and the jacket comprises a secondary lumen through which microbubbles, nanodroplets, and/or pharmaceutical compounds are injectable within a field of the ultrasound energy to enhance ablation of the residual plaque.

According to another aspect of the subject matter described herein, the ultrasound transducer includes a forward-looking stack of piezoelectric elements of the ultrasound transducer for delivering ultrasound energy in an axial direction of the jacket for removing at least a portion of the residual plaque within the stent prior to inserting the ultrasound transducer within the stent.

According to another aspect of the subject matter described herein, a method for therapeutic ablation of residual plaque on, in, and/or surrounding a stent within a blood vessel of a subject is provided. The method includes inserting, into a stent within a blood vessel of a subject, an ultrasound device comprising a jacket and an ultrasound transducer. The method further includes emitting, from at least one active element of the ultrasound transducer, ultrasound energy in a radial direction of the jacket from within the stent and into a target region including residual plaque located on, in, or surrounding the stent. The method further includes ablating a designated portion of the residual plaque in the target region on, in, or surrounding the stent.

According to another aspect of the subject matter described herein, the method includes cooling the ultrasound transducer by flowing a fluid into the jacket and out of the jacket after the fluid has been heated by the ultrasound transducer during cooling the ultrasound transducer.

According to another aspect of the subject matter described herein, the method includes imaging, via an imaging sensor of the ultrasound device, a region external to the jacket for determining a position of the device within the blood vessel of the subject, and guiding, based on the imaging, the ultrasound device through the blood vessel of the subject to the position adjacent to a target region within the blood vessel of the subject.

According to another aspect of the subject matter described herein, inserting the ultrasound device within the stent includes inserting the ultrasound device within a metallic stent and the residual plaque is at least partially within a lumen of the metallic stent.

According to another aspect of the subject matter described herein, the method includes transmitting a signal to the ultrasound transducer to control one or more aspects of the ultrasound emitted energy from the ultrasound transducer via a wired or wireless connection.

According to another aspect of the subject matter described herein, the method includes injecting microbubbles, nanodroplets, and/or pharmaceutical compounds within a field of the ultrasound energy to enhance ablation of the residual plaque.

According to another aspect of the subject matter described herein, the method includes activating a forward-looking stack of piezoelectric elements of the ultrasound transducer to deliver ultrasound energy in an axial direction of the jacket for removing at least a portion of the residual plaque within the stent prior to inserting the ultrasound transducer within the stent.

### BRIEF DESCRIPTION OF THE DRAWINGS

Examples of the subject matter described herein will now be explained with references to the accompanying drawings, of which:

FIG. 1 is a series of diagrams illustrating a procedure for stenting a coronary artery and the occurrence of in-stent restenosis (ISR);

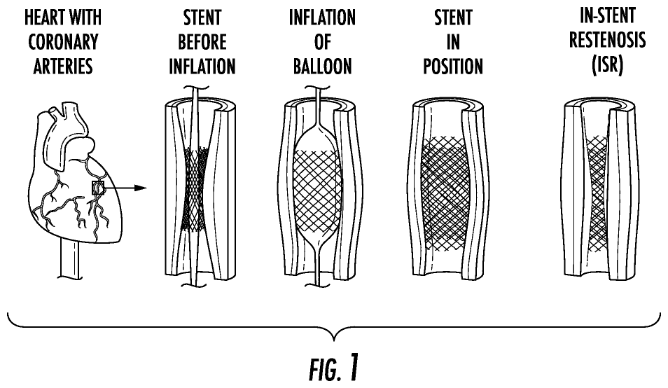

FIG. 2 is a diagram illustrating treatment of residual plaque within a stent using an ultrasound transducer;

FIG. 3 is a diagram illustrating an ultrasound transducer for treatment of ISR from within a stent;

FIG. 4 is a diagram of a catheter including an ultrasound transducer for treatment of ISR from within a stent;

FIG. 5 is an axial view of a blood vessel of a subject illustrating the use of an ultrasound transducer for treatment of ISR from within a stent;

FIGS. 6A and 6B are perspective views of, respectively, an ultrasound transducer comprising a single stack of piezoelectric material and an ultrasound transducer comprising plural stacks of piezoelectric material;

FIG. 7 is a diagram of a blood vessel of a subject illustrating the use of an ultrasound transducer including a forward-looking stack of piezoelectric elements to ablate arterial plaque within a stent prior to inserting the ultrasound transducer within the stent;

FIGS. 8A and 8B are graphs illustrating, respectively, impedance versus frequency and acoustic intensity versus input voltage for a single pillar piezoelectric stack (SPPS) and a multi-pillar piezoelectric stack (MPPS) ultrasound transducer described herein;

FIGS. 8C and 8D are intensity plots of the estimated (e.g., simulated) acoustic pressure fields for the SPPS device of FIG. 6A and the MPPS device of FIG. 6B, respectively;

FIG. 9 is a diagram illustrating the use of a temperature sensor to monitor temperature of a tissue phantom during ablation by an ultrasound transducer as described herein; and

FIG. 10 includes two images of a chicken breast showing results of a tissue ablation experiment using an ultrasound transducer as described herein.

### DETAILED DESCRIPTION

The presently disclosed subject matter now will be described more fully hereinafter, in which some, but not all examples of the presently disclosed subject matter are described. Indeed, the presently disclosed subject matter can be embodied in many different forms and should not be construed as limited to the examples set forth herein; rather, these examples are provided so that this disclosure will satisfy applicable legal requirements.

Focused ultrasound (FUS), or high-intensity focus ultrasound (HIFU), can be used to ablate various malignant bio tissues, such as tumors in the liver, pancreas, and prostate. FUS/HIFU waves vibrate bio molecules in a target region, causing heat due to friction from the induced vibration. The induced heat energy elevates the tissue temperature over 43° C., which causes coagulative necrosis of the bio molecules within the target region. FUS/HIFU devices mostly utilize the surface probe, operating subcutaneously. Such noninvasive FUS/HIFU devices are usually not applicable for organs and vascular systems deep inside a body. The majority of the current FUS/HIFU applications are for noninvasive treatments. Despite the advantages associated with such noninvasive therapeutic methods, the imprecise focusing of the nonintrusive FUS/HIFU has caused many medical doctors to be hesitant to use the common noninvasive modalities for ISR treatment.

Furthermore, the noninvasive FUS/HIFU procedure may yield unwanted tissue damage due to the inaccurately directed focal spot. In contrast, a minimally invasive ultrasound treatment may break through these limitations due to the direct interaction of acoustic waves with the target region. Despite these advantages, therapeutic applications for such intravascular ultrasound transducers are extremely limited due to the small aperture size and the difficulty in the fabrication of actual ultrasound devices having such ultrasound transducers. Current ultrasound transducers are entirely unsuited for clinical use for transmitting a high acoustic energy over the struts of a metallic stent and, furthermore, are incapable of ensuring that the ultrasound energy is focused within a very short focal distance (e.g., about 2-3 mm) to avoid damage to the walls of the blood vessel. Most current devices have a relatively large aperture size (e.g., 2 mm or more), such that they are not preferred to be employed for intravascular applications. Furthermore, acoustic intensity of the current small aperture devices (e.g., 20 W/cm² or less) is not enough to induce necrosis of in-stent neointimal hyperplastic tissue.

Thus, disclosed herein is an intravascular ultrasonic tissue ablation (IVUS-TA) device and technique that have been developed for treatment of ISR by effectively and safely removing in-stent tissues and residual plaques to achieve ISR debulking, as well as acute and long-term luminal gain. Residual plaques within stents are known to exert, in many instances, malignant influence such as ISR. Existing ISR management technologies have multiple technical limitations associated therewith, for example, unpredictable outcomes, imperfect removal of residual plaque, and inaccurate ablation; existing ISR management technologies also have multiple known therapeutic complications, for example, dissection, perforation, bleeding, and recurrence of ISR. Thus, the devices, systems, and methods disclosed herein provide a modality of treatment in which a focused ultrasound technique is utilized to generate ablation (e.g., thermal and/or mechanical) of plaque tissue via a high acoustic intensity and, hence, further development of ISR is effectively suppressed via such devices, systems, and methods.

According to the subject matter disclosed herein, the use of an intravascular ultrasound transducer in an ultrasound device allows for a direct transfer of the acoustic intensity emitted from such transducer to a target region (e.g., within and/or through a stent) that contains residual plaque causing ISR. Due to the direct interaction of acoustic waves on the residual plaque in the target region, the acoustic loss and the required electric power necessary for efficacious treatment of ISR via tissue ablation are significantly reduced compared to currently known noninvasive treatment modalities. Since such an intravascular ultrasound transducer can direct the ultrasound energy (e.g., in the form of an acoustic beam) in a comparatively precise manner, adverse complications as a result of treatment that can arise during and/or after the treatment can be significantly reduced and/or, in some instances, eliminated entirely.

When using conventional ultrasound transducers, it has been found to be difficult to achieve the required acoustic intensity for therapeutic treatment from a small aperture size due to both the insufficient acoustic pressure generated and also the uneven acoustic field. Further complicating any use of such conventional ultrasound transducers for intravascular treatment of ISR is the fact that the presence of a stent barrier further reduces the acoustic power being transmitted from the transducer to a target region. The subject matter disclosed herein can address such disadvantages, for example, via the use of proposed multilayer ultrasound transducers capable of transferring a relatively high acoustic intensity (e.g., about 20 W/cm² or greater) from a miniaturized device (e.g., about 2.0 mm or less in the lateral and the height dimensions) to a target region in which residual plaque is in place over the struts of a stent.

The IVUS-TA technique is enabled by using a novel design of ultrasound transducer(s) for ablative treatment of biological tissue (e.g., vascular plaque). For use in IVUS-TA, such an ultrasound device must be manufactured to have a sufficiently miniaturized size (e.g. about 2 mm or less in diameter) in order to be able to fit within a coronary blood vessel that already has a stent positioned therein. Such ultrasound transducers also must be capable of delivering a high acoustic intensity (e.g., about 20 W/cm² or more) over porous metallic stents (e.g., stents having a mesh-like or net-like structure) into a target region where residual plaque is located. Further, the novel ultrasound transducer disclosed herein is advantageously designed and/or configured to deliver a substantially homogeneous distribution of acoustic intensity over the portion of the target region upon which the ultrasound is incident. Furthermore, providing integrated IVUS imaging for such ultrasound transducers allows targeted ablation between the transducer aperture (e.g., the source from which the ultrasound acoustic energy is emitted) and the biological tissue being treated, accounting for the thickness of the residual plaque and the status of the implanted stent.

Conventional vascular stenting of coronary blood vessels technique generally does not therapeutically treat any volume of plaque that is outside of the volumetric region defined by the stent, which in some instances has been shown to cause further complications after the stent has been inserted. Geometric limitations in the small diameter of a blood vessel restrict the surgical removal of residual plaque using known therapeutic techniques. In addition, medications may, in some instances, result in unexpected bleeding issues when administered at the site of a stent. In contrast, intravascular sonification using ultrasound using the ultrasound transducers and techniques disclosed herein have been found to overcome the limitations of known therapeutic techniques for treatment of ISR. The direct sonification on residual plaque within a target region using such ultrasonic transducers induce coagulative necrosis of neointimal tissue within such residual plaque, which results in the suppression of the further development of the ISR after treatment with the IVUS-TA technique. As long as the ultrasound waves are directed and focused with sufficient precision onto the residual plaque within the target region, it is possible to limit, if not entirely avoid, ablative damage to non-plaque biological tissue, as well as to the vessel wall, adjacent to and/or within the target region, such that such biological structures remain both intact and fully operational (e.g., undamaged, or damaged only minimally, such as will heal without further medical intervention).

Generating high acoustic intensity from a small aperture size of intravascular transducers has been recognized as being technically challenging, since acoustic intensity at the focal zone is proportional to aperture size at a given frequency. Use of a multilayering technique in forming the piezoelectric devices within the ultrasound transducers, however, is now shown to be advantageous in overcoming this previous limitation. Multi-layered piezoelectric materials enable the transmission of high acoustic pressure with respect to unit voltage input due to the reduced electrical impedance level. Additionally, it is advantageous for the operational frequency of the ultrasound transducers disclosed herein for use in the IVUS-TA technique to be relatively low (e.g. about 2 MHz or less) so that the acoustic wave generated by the transducer is not scattered by the structure of the stent due to the acoustic wave having a relatively long wavelength in comparison with the stent wall thickness. For example, acoustic waves produced by piezoelectric devices operating at a frequency of about 1.4 MHz have a wavelength of about 1 mm, which is 10 times greater than the thickness of the stent wall (e.g., about 100 μm). In the case of low-frequency operation, the use of multi-layered piezoelectric devices can be advantageous, since the low resonance frequency does not require the transducer to have relatively thin layers that are typically difficult to fabricate. Examples of such multi-layered piezoelectric devices will now be described in more detail.

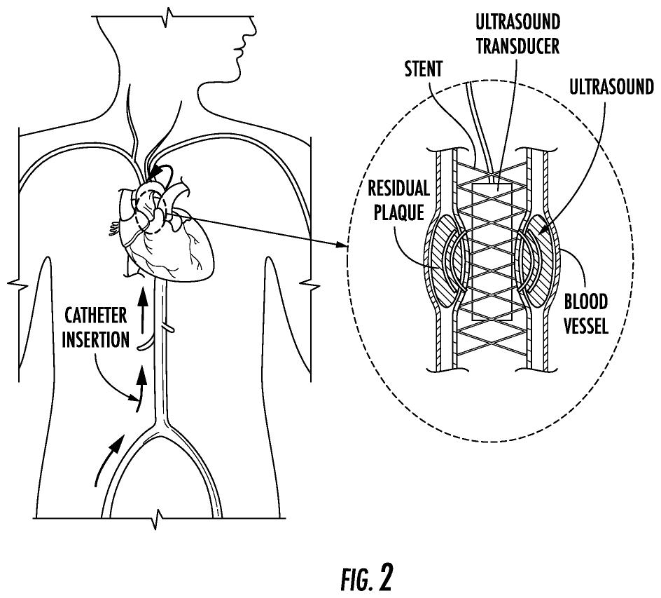

FIG. 2 is a diagram illustrating treatment of residual plaque within a stent using an ultrasound transducer. In FIG. 2, a catheter including an ultrasound transducer is inserted into the blood vessel of a subject, e.g., a human subject, until the ultrasound transducer is positioned within stent. When the ultrasound transducer is positioned within the stent, the ultrasound transducer is activated to deliver ultrasound energy in the axial direction to ablate residual plaque within the blood vessel adjacent to the outer walls of the stent.

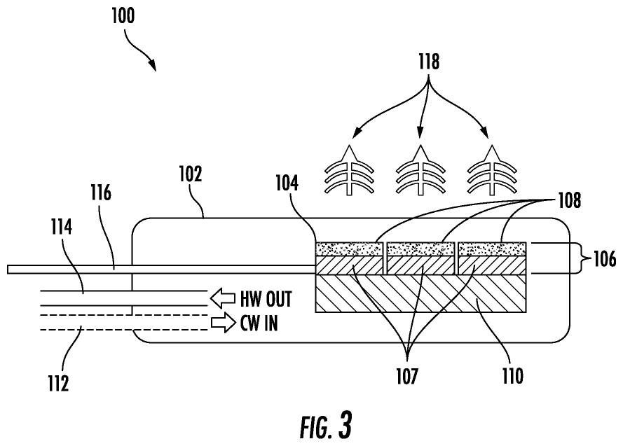

FIG. 3 is a diagram illustrating in more detail example components of an ultrasound device for treatment of residual plaque from within a stent. Referring to FIG. 3, an ultrasound device 100 comprises a cooling jacket 102 surrounding an ultrasound transducer 104. Ultrasound transducer 104 comprise a plurality of stacks 106 of active elements of piezoelectric material. Each stack 106 also includes a matching element or layer 108 for acoustic impedance matching between active elements 107 and the surrounding medium. A common backing layer 110 is positioned under each of the stacks to dampen vibrations. In the illustrated example, each stack 106 includes a single active element or layer 107. In an alternate implementation, each stack 106 may include more than one active element or layer 107.

Jacket 102 includes a cooling liquid inlet 112 to allow a cooling liquid, such as water, to enter jacket 102 and a cooling outlet 114 to allow heated cooling liquid to exit jacket 102. A cable 116 provides signal input, such as the ultrasound excitation signal, to ultrasound transducer 104. In an alternate implementation, the ultrasound excitation signal may be delivered to ultrasound transducer 104 via a wireless connection. In operation, ultrasound device 100 directs ultrasound energy 118 in an axial direction with respect to jacket 102.

In FIG. 3, the MPPS ultrasound transducer comprises of an array (N×M) stacks 106 of piezoelectric material, where the stacks a distributed in the axial or longitudinal direction. In some examples, the array of piezoelectric elements is in the form of an MPPS device, described in greater detail below. Active layer 107 of each stack 106 can comprise, for example, plates made from any suitable piezoelectric material (e.g. PZT-4, PZT-5, PZT-8, and the like). Upon actuation of the active layer, the matching layer, which has a thickness equal to one or more quarter wavelengths at the operating frequency of the ultrasound transducer, intensifies the acoustic pressure output, while backing layer 110 suppresses backpropagating waves. In some examples, backing layer 110 advantageously comprises a light backing material (e.g., air). Active layer 107 is connected to a conductor, such as a cable (e.g., a coaxial cable or other suitable type of cable or wire), which is configured to provide to active layer 107 electric signals (e.g., power-amplified radio frequency (RF) signals).

In FIG. 3, cooling jacket 102 surrounds ultrasound transducer 104. The temperature inside of cooling jacket 102 is modulated by providing a cold fluid (e.g., water at a temperature of 15° C. or lower) into the cooling jacket 102 through inlet tube 112 and then extracting the fluid from cooling jacket 102 via outlet tube 114 after the water has been heated (e.g., via conductive cooling of the ultrasound transducer). Thus, the cold fluid is extracted via outlet tube 114 as a heated fluid due to the actuation of the active layers of the piezoelectric elements. As such, inlet tube 112, cooling jacket 102, and outlet tube 114 define a cooling circuit for the components of the ultrasound device as the fluid circulates through inlet tube 112, cooling jacket 102, and outlet tube 114. Ultrasound transducer 104 delivers a sufficient degree of acoustic intensity (e.g., 20 W/cm² or more) over the target region (e.g., within the stent) to cause tissue necrosis in the targeted residual plaques.

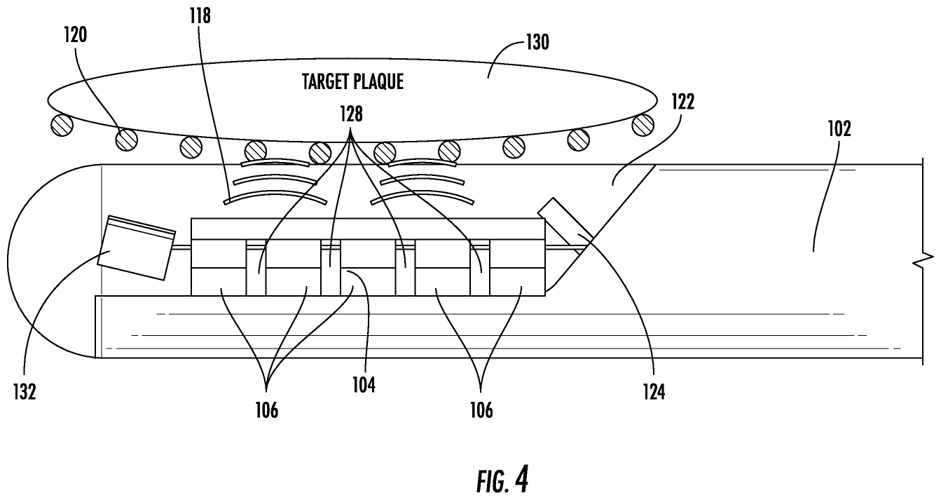

FIG. 4 is a diagram of a catheter including an ultrasound transducer for treatment of ISR from within a stent. In FIG. 4, jacket 102 comprises a catheter for insertion within the lumen of a stent 120, illustrated in FIG. 4 as a sectional view of stent struts. Jacket 102 also serves as an enclosure for containing coolant 122. The coolant inlet and outlet are illustrated schematically in FIG. 4 by coolant circuit 124. Ultrasound transducer 104 comprises a plurality of stacks 106 of piezoelectric material. Each stack 106 is referred to herein as a pillar, and ultrasound transducer 104 illustrated in FIG. 4 is thus referred to as a multi-pillar piezoelectric stack (MPPS) transducer. Adjacent stacks 106 are separated from each other by gaps 128. Gaps 128 may be at least partially filled with an epoxy material or may simply be air gaps. In FIG. 4, stacks 106 are configured to deliver ultrasound energy 118 the radial direction of jacket 102 to ablate plaque from within stent 120 while ultrasound transducer 104 is positioned within stent 120 radially (with respect to the subject's blood vessel) adjacent to plaque 130. Ultrasound transducer 104 further includes an imaging sensor 132, which in the illustrated example is an ultrasound imaging sensor. Imaging sensor 132 may be used to facilitate positioning of ultrasound transducer 104 within the vessel of the subject.

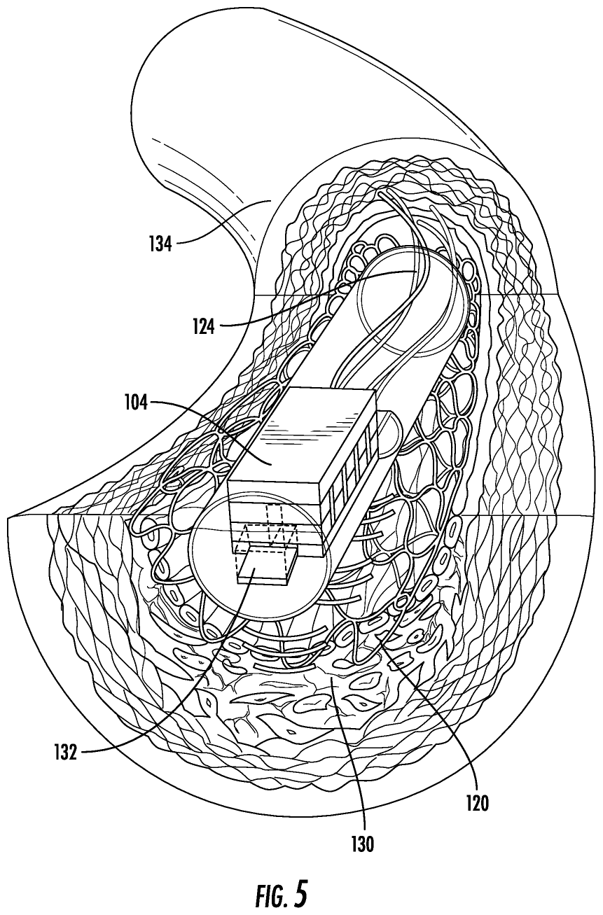

FIG. 5 is an axial view of a blood vessel of a subject illustrating the use of an ultrasound transducer for treatment of ISR from within a stent. In FIG. 5, ultrasound transducer 104 is inserted within blood vessel 134, and, more particularly within stent 120 to ablate plaque 130 allowing stent 120 to expand radially.

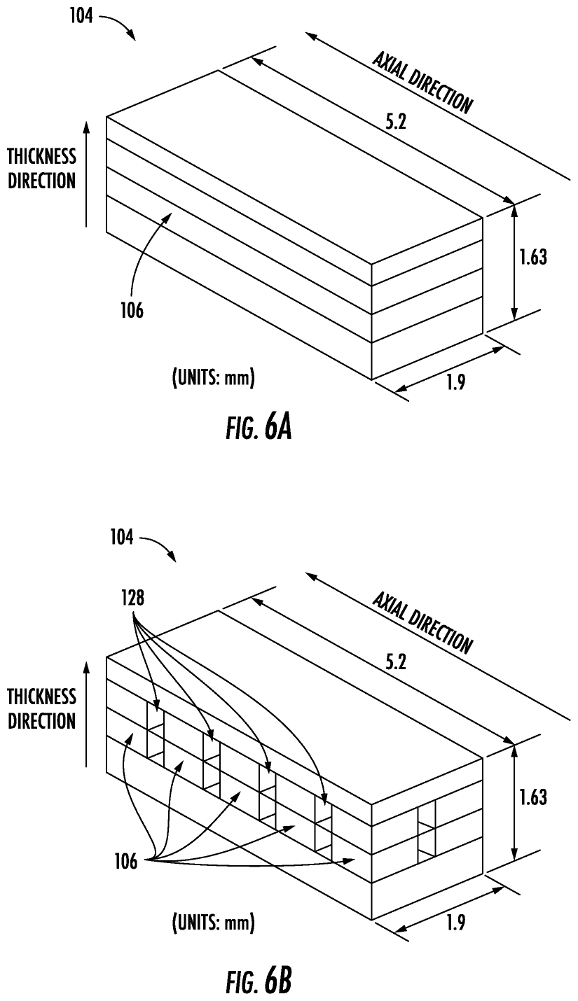

FIGS. 6A and 6B are perspective views of, respectively, an ultrasound transducer comprising a single stack of piezoelectric elements and an ultrasound transducer comprising plural stacks of piezoelectric material. In FIG. 6A, ultrasound transducer 104 includes a single stack 106 of piezoelectric material. In FIG. 6B, ultrasound transducer 104 includes a plurality of stacks 106 of piezoelectric material separated by gaps 128. The dimensions illustrated in FIGS. 6A and 6B are illustrative and the subject matter described herein is not intended to be limited to the illustrated dimensions.

In FIG. 6B, ultrasound transducer 104 comprises a 5×2 array of piezoelectric stacks 106. In one example, the active layer(s) of each of the piezoelectric stacks 106 comprises or consists of PZT-4, a hard piezoelectric material. The thickness of each layer is 400 μm. The lateral gap between adjacent PZT stacks is filled with epoxy resin to suppress the lateral vibration mode of the transducer. The operation frequency is about 1.4 MHz and the matching layer is designed for acoustic impedance matching between the active elements and the surrounding medium at the operating frequency of the ultrasound transducer. In one example, the matching layer is designed to be a quarter wave transformer at the acoustic operating frequency of the ultrasound transducer. Acoustic waves with a relatively long wavelength (e.g., about 1 mm) that are generated by the piezoelectric stacks are not scattered by the struts of the stent. The overall size of the example MPPS transducer shown in FIG. 6B is 1.9 mm (width)×1.63 mm (height)×5.2 mm (length).

The axial, or longitudinal, dimension (e.g., length) of a side-looking ultrasound transducer, as shown in FIGS. 4 and 5, for example, can be larger than the dimension in the radial direction (e.g., diameter, or radius). While this ability for the such a side-looking ultrasound transducer to have a length that is greater than the diameter allows for the aperture size to be increased in the axial direction, it is nevertheless possible for the lateral vibration mode (e.g., in the axial direction) to become coupled together with the thickness mode. Such a coupling between the lateral and thickness vibration modes causes the transducer to operate inefficiently for transmitting a homogeneous acoustic pressure output in the side-looking (e.g., radial) direction. Thus, in order to overcome this technical limitation, a multi-pillar piezoelectric stack (MPPS) ultrasound transducer, shown in FIG. 6B, is disclosed herein. FIGS. 6A and 6B compare the design of the MPPS ultrasound transducer (FIG. 6B) with the design of the SPPS ultrasound transducer (FIG. 6A). Gaps 128 (e.g., epoxy) are provided (e.g., filled in) between adjacent piezoelectric stacks to suppress the transmission of lateral vibration modes between adjacent piezoelectric stacks to prevent vibrational coupling between such adjacent piezoelectric stacks, such that the piezoelectric stacks of the ultrasound transducer (and, therefore, also the ultrasound transducer itself) predominantly vibrate in the thickness, or radial, direction. The use of MPPS ultrasound transducers in the ultrasound devices disclosed herein is advantageous, at least because the use of such MPPS devices provides effective targeting of acoustic intensity upon the aperture surface, a substantially homogeneous acoustic pressure field on, over, about, and/or around a targeting region, and an even and broad therapeutic range.

Intravascular ultrasound (IVUS) imaging allows for scanning to determine geometric information of residual plaque within a stent and/or monitoring the status (e.g., position, size, shape, etc.) of residual plaque at a target region during therapeutic treatment (e.g., via sonification using the ultrasound transducers disclosed herein). The examples of the ultrasound devices shown in FIGS. 4 and 5 show an example position for an IVUS image sensor to perform such scanning. Due to the typically small size of residual plaque, the imaging sensor advantageously has a fine resolution and a high sensitivity. In one example, the IVUS imaging sensor can be a high-frequency (e.g., about 20 MHz or higher) side-looking ultrasound imaging transducer, which can be designed and fabricated using a piezo-composite micromachined ultrasound transducer technique. In the examples shown, the IVUS imaging sensor is assembled in a housing formed by an outer jacket (e.g., a catheter), together with the piezoelectric transducers of the ultrasound transducer that are used for therapeutic sonification of residual plaque. Thus, the total lumen size of the ultrasound device advantageously does not exceed 4-6 Fr. (French gauge size), such that the ultrasound device can penetrate into (e.g., positioned within) the middle of the stent lumen.

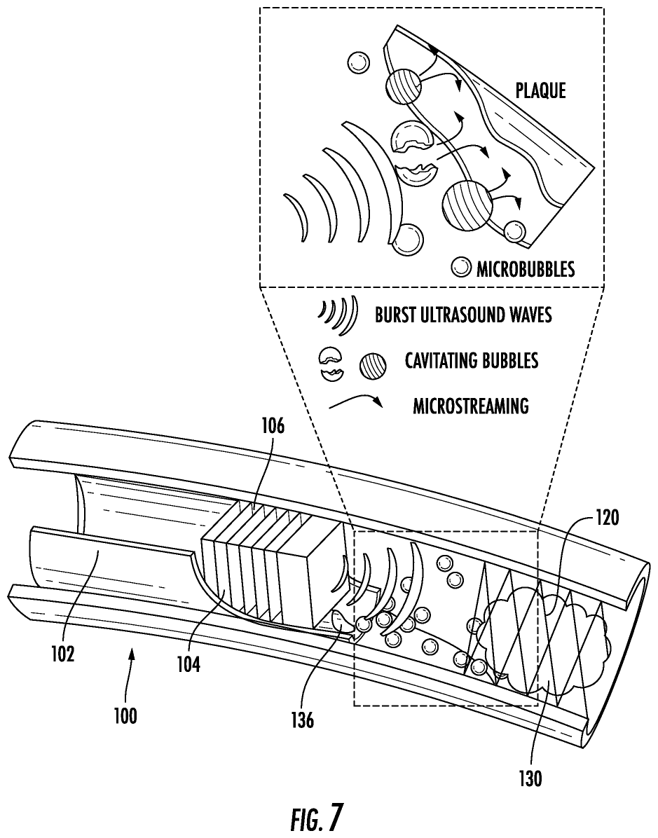

FIG. 7 shows an example of a forward-looking, or axially-directed, piezoelectric device that can be provided within the example ultrasound device shown in FIGS. 4 and 5 (e.g., positioned axially beyond the IVUS imaging sensor). By “forward-looking”, it is meant that the ultrasound transducer includes at least one stack of piezoelectric elements configured to deliver ultrasound energy focused maximally in the axial direction of the blood vessel of the patient or subject (or the jacket of the ultrasound transducer). In contrast, the “side-looking” ultrasound transducers illustrated in FIGS. 4 and 5 comprise stacks of piezoelectric material configured to deliver ultrasound energy focused maximally in the radial direction of the subject's blood vessel or the jacket of the ultrasound transducer. The forward-looking transducer and side-looking transducers can work independently in a separate mode or in a combined mode as needed.

In FIG. 7, ultrasound device 100 includes an ultrasound transducer 104 having a single stack 106 of piezoelectric elements oriented axially within jacket 102 to deliver ultrasound energy in the axial direction within the subject's blood vessel to target plaque 130 within stent 120 prior to insertion of ultrasound transducer 104 within stent 120. Ultrasound device 100 further includes a therapeutic and cavitation-enhancing agent injection lumen 136 for injecting therapeutic and cavitation-enhancing agents into stent 120. In the illustrated example, the cavitation-enhancing agents include microbubbles and/or nanodroplets that penetrate plaque 130 and cavitate, change phase, oscillate in diameter, and/or burst to disrupt plaque 130 when ultrasound transducer 104 applies ultrasound energy. Tissue plasminogen activator (tPA) is one example of a therapeutic agent that ultrasound device 100 can deliver via lumen 136 to plaque 130 within stent 120. Thus, in FIG. 7, jacket 102 may form a primary lumen in which ultrasound transducer 104 is located, and therapeutic and cavitation-enhancing agent injection lumen 136 may comprise a secondary lumen located within the primary lumen for the injection of therapeutic and/or cavitation-enhancing agents.

The inclusion of such a forward-looking piezoelectric device in such side-looking ultrasound transducers as are disclosed herein can be advantageous, for example, in therapeutic treatment (e.g., removal) of residual plaque within a stent that may occur, for example, when the intrusion of the residual plaque within the stent reduces the lumen size (e.g., effective diameter) within the stent such that the diameter of the ultrasound device is greater than the lumen size of the stent designated for therapeutic ablative treatment of ISR. Thus, the forward-looking piezoelectric device can be used initially to reduce the size of the residual plaque to a size that allows for passage of the ultrasound device (e.g., the outer contour of which is defined by the outer jacket in which the ultrasound transducer is contained) through the lumen of the stent, such that the ablative therapeutic treatment of the residual plaque can then be performed by the side-looking piezoelectric devices. Additionally, such a forward-looking piezoelectric device can be used for generating sonic energy that will pop microbubbles for the targeted administration of medication delivered, for example, via a microbubble-injection tube, as shown in FIG. 7, either before or after the therapeutic ablative ISR treatment via the side-looking piezoelectric devices.

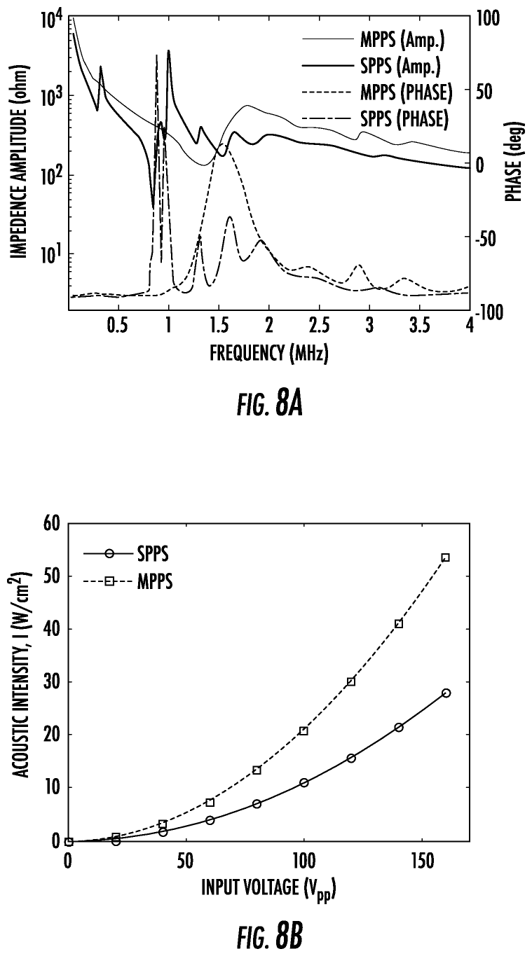

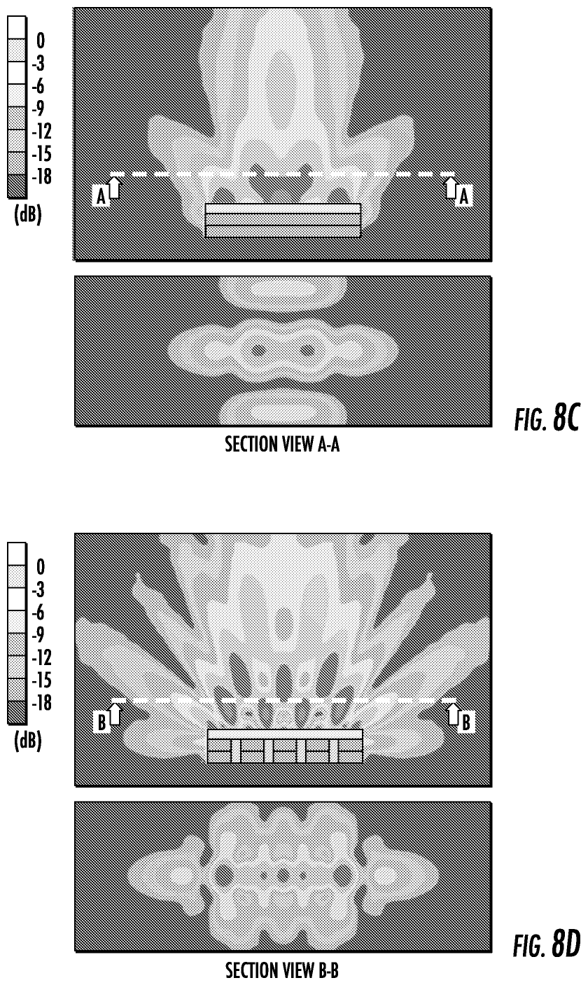

FIGS. 8A-8D show the results obtained from numerical simulation of the MPPS device for the validation of the design; the numerical simulation was conducted using the commercialized software, ANSYS (v. 17.1, Cannonsburg, PA). FIG. 8A is a graphical plot showing the simulated electric impedance responses of the SPPS transducer of FIG. 6A with that of the MPPS transducer of FIG. 6B. As can be seen from the results, multiple vibration modes were coupled together in the SPPS device of FIG. 6A, while the MPPS device of FIG. 6B exhibited predominantly vibration in the thickness mode at 1.4 MHz. The impedance level at the resonance frequency was about 131Ω. FIGS. 8C and 8D are intensity plots of the estimated (e.g., simulated) acoustic pressure fields for the SPPS device FIG. 6A and the MPPS device of FIG. 6B, respectively.

As can be seen by comparing the simulated acoustic pressure field obtained using the SPPS device of FIG. 6A with that of the MPPS device of FIG. 6B, the acoustic pressure field produced by the SPPS device was comparatively narrower than that which was produced by the MPPS device, primarily if not entirely due to the coupling of the lateral and thickness vibration modes of the piezoelectric stack in the SPPS device, whereas the MPPS device can generate, as shown in FIG. 8D, a substantially evenly-distributed acoustic pressure field in the thickness direction. Furthermore, as shown in FIG. 8B, the acoustic intensity (spatial-average temporal-average) of the MPPS device is about 90% greater than that of the SPPS device. Additionally, the mechanical index produced by the MPPS device was estimated, based on the simulation results, to be about 1.49 at 100 Vₚₚ input voltage.

The comparatively higher mechanical index level and the direct interaction with (e.g., sonification of) biological tissue (e.g., residual plaque) within a target region causes inertial cavitation of the biological tissue, which induces the ablation effect upon insonification. The specifications of the example MPPS device illustrated and described herein are shown in the table below.

| Operating | | Input | |
|---|---|---|---|
| Frequency | 1.4 MHz | voltage | 100 Vₚₚ |
| Duty Cycle | 40% | Beam Width | 4.6 × 1.0 |
| | | at 1 mm | mm² |
| Peak-to- | 2.49 MPa | Mechanical | 1.49 |
| Peak | | Index | |
| pressure | | | |
| Acoustic | 20.9 W/cm² | | |
| Intensity | | | |

The ultrasound transducer illustrated in FIG. 4 was fabricated based on simulation results. In FIG. 4, each stack 106 includes two layers of PZT-4, each having a thickness of about 400 μm. The layers were bonded together via a layer of conductive silver epoxy (E-Solder 3021, Von-Roll Inc., Cleveland, OH) to form each of the piezoelectric elements, or stacks 106, of the ultrasound device. Once the epoxy used for each stack 106 bonding was fully cured, a matching layer (e.g., having a construction that was unitary, monolithic, continuous, uninterrupted, etc.) comprising a composite of epoxy and Al₂O₃ was attached on top of all of piezoelectric stacks 106, also via the conductive silver epoxy by which the two layers of PZT-4 were bonded in each of piezoelectric stacks 106. The kerf width, to fill in the gaps between adjacent piezoelectric stacks 106 with the passive material, was 300 μm. Using a dicing machine (DAD323, Disco Corp., Tokyo, Japan), the kerf can be sliced partially, with the matching layer part remained intact. In this example, the passive material was an epoxy resin (EPO-TEK 301, Epoxy Tech. Inc., Billerica, MA). After the epoxy resin that formed the passive material was sufficiently cured, the side surfaces were sliced through. Thus, in the ultrasound device shown in FIG. 4, an MPPS ultrasound transducer is used. Next, the MPPS device was coated by an about 10 μm thick encapsulation layer comprising Parylene-C. Finally, the encapsulated MPPS device was assembled in outer jacket 102 (e.g., a catheter), including coolant circuit 124.

IVUS imaging sensor 132 was also integrated within jacket 102. IVUS imaging sensor 132 in the examples disclosed herein has a center frequency of about 40 MHz, a reasonable sensitivity (e.g., 100 mV or more), and a wide fractional bandwidth (e.g., 80% or more). To meet the requirements, PMN-PT 1-3 composite material, known for having a relatively high piezoelectric constant and electromechanical coupling, can be used for IVUS imaging sensor 132. In the instant example, IVUS imaging sensor 132 is in the form of a piezoelectric device, thus comprising a backing layer, one or more active layers, and a matching layer. The matching layer of IVUS imaging sensor 132 is designed to be a quarter wavelength in thickness at the operating frequency of IVUS imaging sensor 132 using an Al₂O₃/epoxy composite material. The backing layer comprises or consists of a titanium/epoxy composite material to suppress reverberation and/or vibration. IVUS imaging sensor 132 is integrated in jacket 102 (e.g., a catheter) together with the MPPS ultrasound transducer in the ultrasound device shown in FIG. 4.

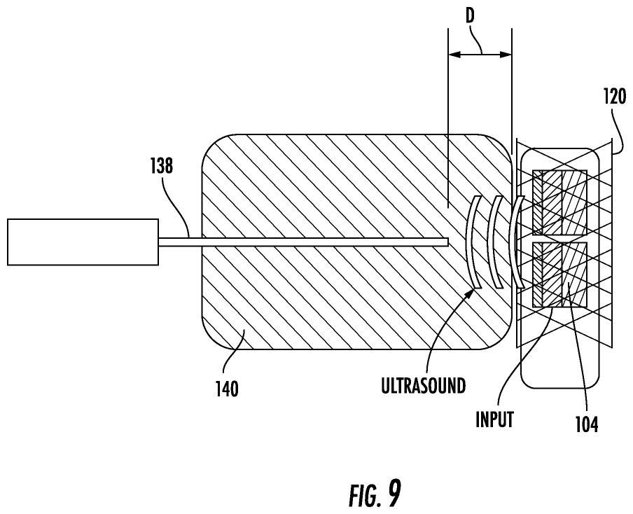

In one example, the ultrasound device disclosed herein is configured to deliver an acoustic intensity of 30 W/cm² or more at a specific distance (see, e.g., the distance D in FIG. 9) from the aperture (e.g., ˜1 mm) to ablate a target tissue without damaging the blood vessel adjacent to the target tissue. The high frequency (e.g., 50 MHZ) IVUS imaging probe will provide a relatively high-resolution ultrasound-based image of a target region.

The ultrasound devices described herein can be used to implement a method for elimination of in-growth of tissue (e.g., residual plaque) within a stent for increasing the luminal area thereof. Such methods provide improved blood flow and also suppress further in-growth of such tissues after the initial stenting operation via focused ultrasonic acoustic energy (e.g., waves) produced by a side-looking intravascular high-intensity focused ultrasound (HIFU) transducer, examples which are shown in FIGS. 2-5. The ultrasound device is guided to the target position within the stent (e.g., a metallic stent) and, thus, to the residual plaque that is at least partially positioned between the stent and the wall of the blood vessel. The positioning of the ultrasound device can be achieved using, for example, real-time fluoroscopy, magnetic resonance imaging (MRI), computer tomography (CT), via the IVUS imaging sensor, or the like. After confirming that the ultrasound device is correctly positioned at the target region within the stent, the ultrasound transducer of the ultrasound device generates ultrasound acoustic energy in the radial direction of the ultrasound transducer. The ultrasound delivered to the target region ablates the residual plaque and suppresses further in-growth of the residual plaque within the stent.

In contrast to typical HIFU therapeutic treatments, the HIFU produced for the treatment of residual plaque that causes ISR must be capable of demonstrating clinical safety, as well as efficacy. The clinical target (e.g., plaque tissue) is typically confined within a narrow volumetric region between the stent and the blood vessel, this volumetric region typically having a thickness of between about 1-2 mm, inclusive. Given the need to avoid ablative damaging of the walls of the blood vessel, it is necessary to demonstrate that an effective sonification area can be maintained within a sufficiently small distance from the aperture of the ultrasound transducer. As such, an example of how such a characterization of the ultrasound transducer for ablative therapeutic treatment of ISR can be performed is shown schematically in FIG. 9. As shown in FIG. 9, the effective therapeutic treatment distance is designated by the distance (D) between the aperture of ultrasound transducer 104 and the tip of a temperature sensor 138 (e.g., a thermocouple). After the appropriate distance has been determined, a tissue phantom 140 compatible with biological plaque tissues is positioned around temperature sensor 138, including between ultrasound transducer 104 and temperature sensor 138. It has been determined that, in order to ensure clinical safety during use, the temperature detected by temperature sensor 138 at the distance D from ultrasound transducer 104 cannot be greater than 43° C. during sonification, which corresponds to a temperature at which human biological tissues begin to suffer damage due to ablation. Thus, if temperature sensor 138 does not detect a temperature above 43° C., the safety outside of the target region can be ensured. In order to correlate the temperature of tissue phantom 140 with that of living human tissue, it is advantageous to ensure that tissue phantom 140 be preheated to the same temperature the temperature within a human body. While the distance D may, for some examples, be about 3 mm, the distance D can be manually selected by the user based on the intended clinical application. The ultrasonic “dose” emitted by the ultrasound transducer is generally determined by the input voltage and the duty cycle. This gives the specification of the required acoustic intensity. Next, the temperature can be further tuned changing the pulse repetition frequency in the range below 1 kHz, under the chosen acoustic intensity condition.

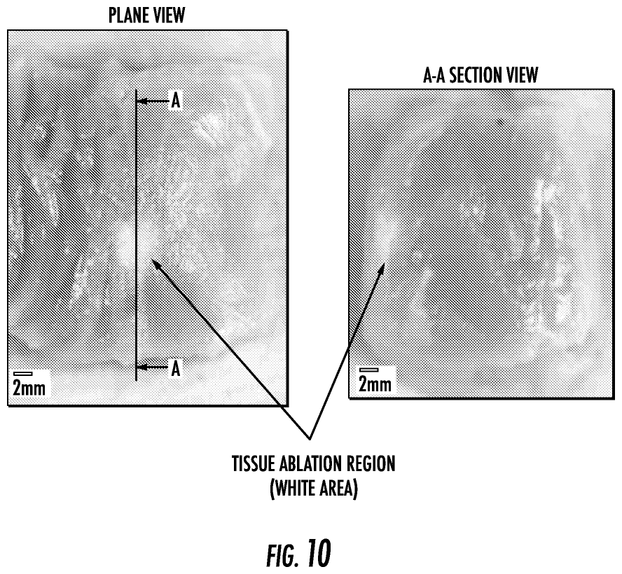

FIG. 10 is an illustration of successful ablation of biological tissue utilizing an example ultrasound device disclosed herein. In FIG. 10, the biological tissue is chicken breast and the white (e.g., discolored) portion of the chicken breast, which is generally comparable to vascular plaque tissue, is the region in which cell necrosis was induced within the chicken breast due to a hyperthermia effect induced by the ultrasound emitted by the ultrasound transducer of the ultrasound device.

The presently disclosed subject matter addresses several critical industrial/commercial problems existing in the conventional ultrasound therapeutic modalities. The small package of the ultrasound devices disclosed herein, which incorporate a cooling circuit therein for cooling of an ultrasound transducer, is advantageous for guiding through blood vessels into the pre-installed metallic stent. The example ultrasound devices disclosed herein are capable of delivering a sufficiently high acoustic intensity through metallic stents to ablate plaque tissues placed over the stent struts, such that additional growth of the residual plaque is restricted, which will consequently reduce the reoccurrence rate of ISR, which is economically beneficial compared to repeated stenting surgeries and will benefit more patients as a result thereof.

The terminology used herein is for the purpose of describing particular examples only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a component” includes a plurality of such components, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some examples ±20%, in some examples ±10%, in some examples ±5%, in some examples ±1%, in some examples ±0.5%, and in some examples ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C and D.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A device for therapeutic ablative treatment of residual plaque on, in, or surrounding a stent within a blood vessel of a subject, the device comprising:
a jacket configured for insertion within a stent within a blood vessel of the subject; and
an ultrasound transducer located within the jacket and having at least one active element oriented to deliver ultrasound energy in a radial direction of the jacket from within the stent to ablate residual plaque located on, in, or surrounding the stent.

2. The device of claim 1, wherein the ultrasound transducer comprises one or more piezoelectric elements.

3. The device of claim 2, wherein the one or more piezoelectric elements comprise a plurality of stacks of piezoelectric material, each stack forming a pillar, and the pillars forming a multi-pillar piezoelectric stack (MPPS) device.

4. The device of claim 3, wherein each of the pillars of the MPPS device is separated from adjacent pillars by a gap for reducing lateral vibrational coupling between adjacent pillars.

5. The device of claim 4, comprising an epoxy resin, wherein each of the gaps is filled with the epoxy resin.

6. The device of claim 3, wherein each of the piezoelectric elements comprises at least one active layer.

7. The device of claim 6, wherein the MPPS device comprises a common backing layer and a common matching layer, between which the at least one active layer of each of the piezoelectric elements is positioned.

8. The device of claim 1, comprising a cooling circuit for cooling the ultrasound transducer, the cooling circuit comprising an inlet tube for providing a flow of a fluid into the jacket and an outlet tube for providing a flow of the fluid out of the jacket after the fluid has been heated by the ultrasound transducer during cooling the ultrasound transducer.

9. The device of claim 1, comprising an imaging sensor for imaging an area external to the jacket for positioning the device adjacent to a target region within the blood vessel of the subject.

10. The device of claim 9 wherein the imaging sensor comprises an ultrasound imaging sensor.

11. The device of claim 1, wherein:
the stent is a metallic stent and the residual plaque is at least partially within a lumen of the metallic stent; and
the jacket comprises a catheter.

12. The device according to claim 1, wherein the ultrasound transducer is configured to receive a signal via a wired or wireless connection for controlling one or more aspects of the ultrasound energy emitted from the ultrasound transducer.

13. The device according to claim 1, wherein:
the jacket comprises a primary lumen and a secondary lumen that each extend longitudinally within the jacket; and
the jacket comprises a secondary lumen through which microbubbles, nanodroplets, and/or pharmaceutical compounds are injectable within a field of the ultrasound energy to enhance ablation of the residual plaque.

14. The device according to claim 1, wherein the ultrasound transducer includes a forward-looking stack of piezoelectric elements of the ultrasound transducer for delivering ultrasound energy in an axial direction of the jacket for removing at least a portion of the residual plaque within the stent prior to inserting the ultrasound transducer within the stent.

15. A method for therapeutic ablation of residual plaque on, in, and/or surrounding a stent within a blood vessel of a subject, the method comprising:
inserting, into a stent within a blood vessel of a subject, an ultrasound device comprising a jacket and an ultrasound transducer;
emitting, from at least one active element of the ultrasound transducer, ultrasound energy in a radial direction of the jacket from within the stent and into a target region including residual plaque located on, in, or surrounding the stent; and
ablating a designated portion of the residual plaque in the target region on, in, or surrounding the stent.

16. The method of claim 15, wherein the ultrasound transducer includes one or more piezoelectric elements.

17. The method of claim 16, wherein the ultrasound transducer includes a plurality of stacks of piezoelectric material, where each stack forms a pillar, and the pillars form a multi-pillar piezoelectric stack (MPPS) device.

18. The method of claim 17, wherein adjacent pillars are separated from each other by a gap to reduce lateral vibrational coupling between adjacent pillars.

19. The method of claim 18, wherein each gap is filled with an epoxy resin.

20. The method of claim 17, wherein each of the piezoelectric elements comprises at least one active layer.

21. The method of claim 20, wherein the MPPS device comprises a common backing layer and a common matching layer, between which the at least one active layer of each of the piezoelectric elements is positioned.

22. The method of claim 15, comprising cooling the ultrasound transducer by flowing a fluid into the jacket and out of the jacket after the fluid has been heated by the ultrasound transducer during cooling the ultrasound transducer.

23. The method of claim 15, comprising:
imaging, via an imaging sensor of the ultrasound device, a region external to the jacket for determining a position of the device within the blood vessel of the subject; and
guiding, based on the imaging, the ultrasound device through the blood vessel of the subject to the position adjacent to a target region within the blood vessel of the subject.

24. The method of claim 23, wherein imaging via the imaging sensor includes imaging via an ultrasound image sensor.

25. The method of claim 15, wherein:
inserting the ultrasound device within the stent includes inserting the ultrasound device within a metallic stent and the residual plaque is at least partially within a lumen of the metallic stent; and
the jacket comprises a catheter.

26. The method of claim 15, comprising transmitting a signal to the ultrasound transducer to control one or more aspects of the ultrasound emitted energy from the ultrasound transducer via a wired or wireless connection.

27. The method of claim 15, comprising injecting microbubbles, nanodroplets, and/or pharmaceutical compounds within a field of the ultrasound energy to enhance ablation of the residual plaque.

28. The method according to claim 15 comprising activating a forward-looking stack of piezoelectric elements of the ultrasound transducer to deliver ultrasound energy in an axial direction of the jacket for removing at least a portion of the residual plaque within the stent prior to inserting the ultrasound transducer within the stent.
